(19) United States
(12) Patent Application Publication
(10) Pub. No.: US 2013/0178921 A1
(43) Pub. Date: Jul. 11, 2013

(54) SYSTEM AND APPARATUS PROVIDING A CONTROLLED LIGHT SOURCE FOR MEDICINAL APPLICATIONS

(71) Applicant: CHARLES BRIAN ROGERS, Dunedin, FL (US)

(72) Inventor: CHARLES BRIAN ROGERS, Dunedin, FL (US)

(21) Appl. No.: 13/783,387

(22) Filed: Mar. 3, 2013

Related U.S. Application Data
(63) Continuation of application No. 11/686,767, filed on Mar. 15, 2007.

(51) Int. Cl. A61N 5/06

(52) U.S. Cl. CPC A61N 5/0624
USPC 607/92; 607/90

(57) ABSTRACT

An application for a light source for killing blood pathogens. The light source includes multiple ultraviolet light emitting diodes and a visible-spectrum light emitting diode. A light mixer combines light from the ultraviolet light emitting diodes and the visible-spectrum light emitting diode and focuses a mixed light into a fiber optic for delivery to an intravenous needle. A controller adjusts an amount of current delivered to the ultraviolet light emitting diodes and visible-spectrum light emitting diode. A touch screen is interfaced to the controller for inputting commands and a display is interfaced to the controller for outputting information.

### CROSS-REFERENCE TO RELATED APPLICATIONS

This application is a continuation of U.S. Pat. App. Ser. No. 11/686,767 (filed Mar. 15, 2007) and said document is hereby incorporated by reference in its entirety.

### FIELD OF THE INVENTION

This invention relates to the field of using light rays to kill pathogenic organisms and more particularly to a system and apparatus for emitting ultraviolet and visible light at controlled intensities.

### BACKGROUND OF THE INVENTION

It is well known to use ultraviolet light (UV) to kill pathogens in a liquid such as water. Many systems exist to expose liquids to ultraviolet light with the object of destroying pathogens. Additionally, it is well know to guide fiber optic instruments into arterial blood vessels. U.S. Pat. No. 4,830,460 to Goldenberg describes using ultraviolet light laser energy to ablate atherosclerotic plaque. U.S. Pat. No. 5,053,033 to Clarke describes an optical fiber for delivering ultraviolet light radiation to a blood vessel site following angioplasty to kill aortic muscle cells at the sight. U.S. Pat. No. 6,117,128 to Gregory describes a source of laser energy coupled to an optical fiber that is transported by a catheter to treat vascular thrombosis disorders in the brain. U.S. Pat. No. 6,187,030 to Gart describes a flexible fiber optic bundle connected to a light source for the treatment of internal and external diseases.

U.S. Pat. No. 6,908,460 to DiStefano describes an apparatus for conveying light through an intravenous needle to kill blood pathogens and is hereby incorporated by reference. This patent describes using a combination of ultraviolet light and visible light (e.g., white light) alternately though an optical fiber and into a patient's venous system to kill pathogens in the venous system. The ultraviolet light kills pathogens such as bacteria, virus, fungi, molds and other unclassified pathogens. This patent describes a treatment of exposure to ultraviolet light of 200 to 450 nanometers in wavelength for around 30 minutes and exposure to visible light of 450 to 1100 nanometers in wavelength for another 30 minutes. This patent does not describe a method or apparatus for generating the desired wavelengths of light, nor for controlling the energy levels and duration of the light.

What is needed is an apparatus that will generate a selected wavelength of light at a selected power level for a specified duration of time.

### SUMMARY OF THE INVENTION

In one embodiment, a light source for killing blood pathogens is disclosed including at least two light emitting diodes and a device for combining light from the light emitting diodes into a mixed light and focusing the mixed light into a fiber optic for delivery to an intravenous needle. A controller is provided for programmatically controlling the light emitting diodes and has an input device for inputting commands and an output device for displaying information.

In another embodiment, a light source for killing blood pathogens is disclosed including ultraviolet light emitting diodes and a visible-spectrum light emitting diode. A light mixer combines light from the ultraviolet light emitting diodes and the visible-spectrum light emitting diode and focuses a mixed light into a fiber optic for delivery to an intravenous needle. A controller adjusts an amount of current delivered to the ultraviolet light emitting diodes and visible-spectrum light emitting diode. A touch screen is interfaced to the controller for inputting commands and a display is interfaced to the controller for outputting information.

In another embodiment, a light source for killing blood pathogens is disclosed including ultraviolet light emitting diodes, each emitting light at a different wavelength and a visible-spectrum light emitting diode. A light mixer combines light from the ultraviolet light emitting diodes and the visible-spectrum light emitting diode and focuses the light into a fiber optic for delivery to an intravenous needle. A controller adjusts the amount of current delivered to the ultraviolet light emitting diodes and to the visible-spectrum light emitting diode. A minority of the light is reflected onto a photodiode which is coupled to the controller. A touch screen is provided for inputting commands and a display for outputting information.

### BRIEF DESCRIPTION OF THE DRAWINGS

The invention can be best understood by those having ordinary skill in the art by reference to the following detailed description when considered in conjunction with the accompanying drawings in which:

FIG. 1 illustrates a block diagram of a controller of the present invention.

FIG. 2 illustrates a schematic view of the light sources of the present invention.

FIG. 3 illustrates an isometric view of a typical enclosure for the present invention.

FIG. 4 illustrates an isometric view of the interrelationship between the light sources, photo detector and fiber optics of the present invention.

### DETAILED DESCRIPTION OF THE INVENTION

Reference will now be made in detail to the presently preferred embodiments of the invention, examples of which are illustrated in the accompanying drawings. Throughout the following detailed description, the same reference numerals refer to the same elements in all figures.

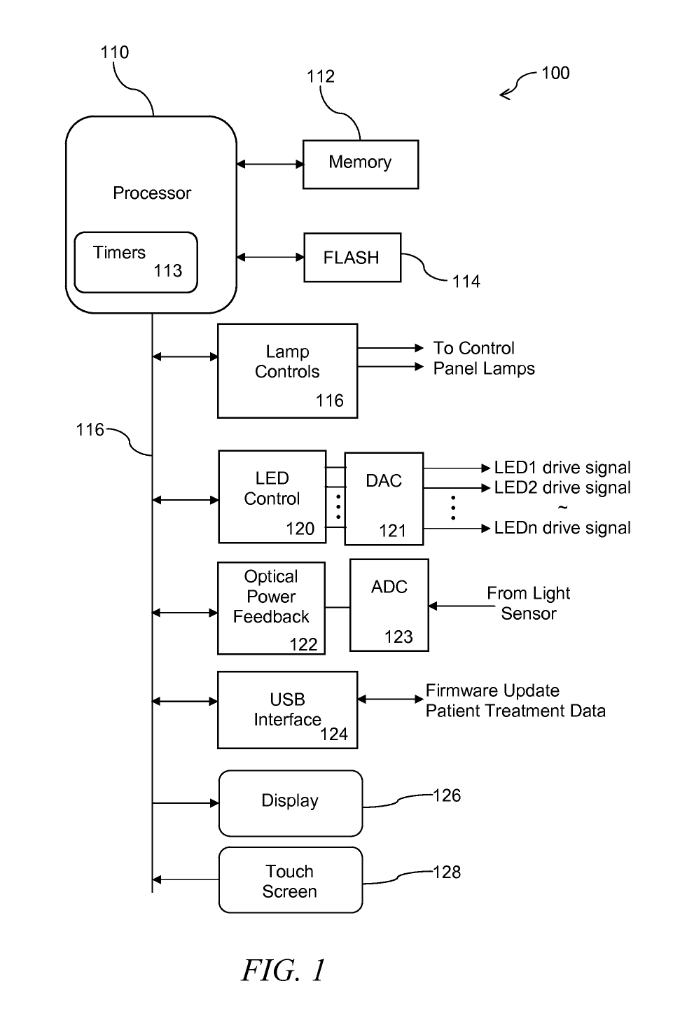

Referring to FIG. 1, a block diagram of a controller of the present invention is shown. This system is designed to deliver user selectable optical power at user selectable wavelengths delivered to the patient via, for example, a high performance UV transmitting fiber optic cable, preferably a silica fiber optic cable. The system is configured to provide a single or multiple concurrent treatments. The sources of light are preferably solid state LEDs (Light Emitting Diodes) emitting light at their fundamental wavelengths. In the preferred embodiment, there are four ultraviolet LEDs delivering light power in the high-UVB and UVA portion of the spectrum, (290 nm-365 nm). Also in the preferred embodiment, visible energy is emitted by a separate LED which delivers light with wavelengths of from 450 nm to 750 nm.

The controller 100 has a processor 110 which can be any microprocessor or controller such as an Intel 80C51 or the like. In some embodiments, the processor uses external memory 112 to store data and instructions while in other embodiments, the processor has imbedded memory while in still other embodiments, both external memory 112 and internal memory are used. In the preferred embodiment, programs (firmware) are stored in persistent memory 114 until they are executed after loading them in memory 112. There are many forms of persistent memory 114 that are possible including, but not limited to, flash, ROM, EPROM, EEPROM, magnetic storage, etc. The processor communicates with input/output devices through a bus 116.

A set of output bits coupled to the bus 116 are used to control various lamps 116 and other indicia. For example, indicator LEDs or lamps on the front panel indicate power on (e.g., green), ultraviolet treatment active (e.g., Blue) and visible light treatment (e.g., white led). In the preferred embodiment, operator input is accepted from a touch screen 128 and operator display communications are presented on a display 126, preferably a graphics display such as a liquid crystal display (LCD). To communicate with the outside world, an interface, such as a universal serial bus (USB) interface 124, is provided. This USB interface 124 is used, for example, to load/reload/update firmware and to transfer patient treatment data.

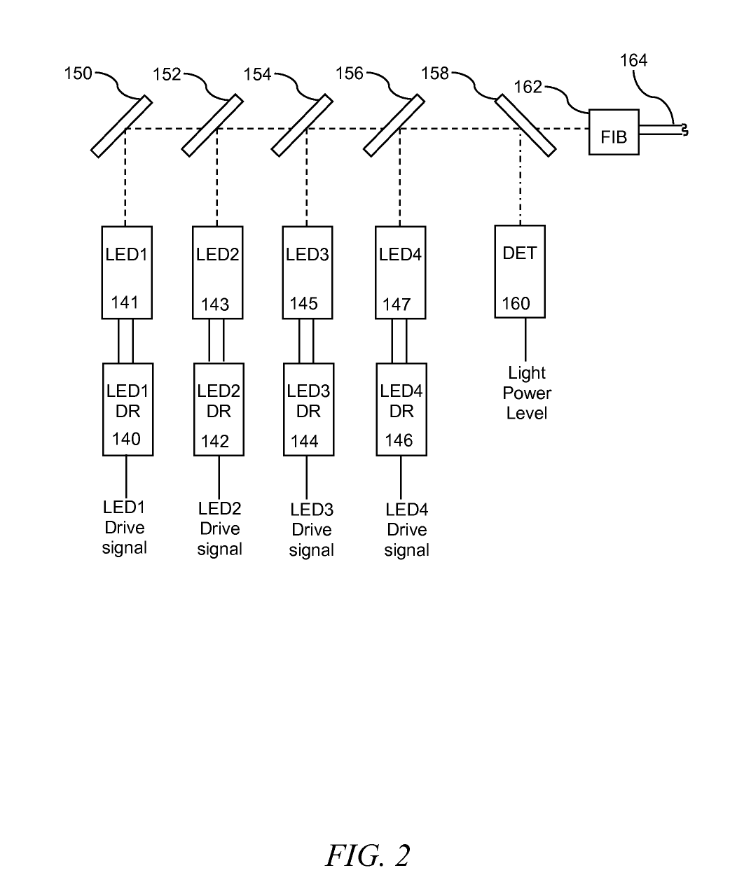

Being that the light output from the present invention is injected into a living creature, it is important that the wavelength, optical power output and duration be tightly controlled. The wavelength is controlled by selecting one or more ultraviolet and visible light emitting diodes 141/143/145/147 (see FIG. 2), each having a light output at a fundamental wavelength. In one embodiment, each LED 141/143/145/147 is encapsulated in a separate package. In other embodiments, some of the LEDs 141/143/145/147 are encapsulated in a common package while other LEDs 141/143/145/147 are encapsulated in different packages. In other embodiments, all of the LEDs 141/143/145/147 are encapsulated in one common package.

The controller 100, under program control, adjusts the optical power output of each light emitting diode through a set of LED control output ports 120 that are coupled to one or more digital to analog converters (DACs) 121. The outputs of the DACs 121 drive the light emitting diodes 141/143/145/147 though current or voltage drivers 140/142/144/146 (see FIG. 2). The duration is controlled by timers 113 internal to the processor 110 of the controller.

Because of manufacturing variance and temperature-related variances, the optical power output is not deterministic based upon the current delivered to the LED(s) 141/143/145/147. To better control the optical power output, the light output of the LED(s) is monitored with an optical sensor 160 (see FIG. 2) such as a photodiode or the like. The signal from the optical sensor is converted to digital by an analog to digital (ADC) converter 123 and inputted to the processor 110 through an input port 122. In this way, the processor 110 monitors the optical power output and adjusts the output values delivered to the LED control 120 when the optical power exceeds or under runs the desired optical power output level.

Referring now to FIG. 2, a schematic view of the light sources and current drivers of the present invention will be described. Each LED 141/143/145/147 is driven by a LED driver 140/142/144/146. LED drivers are well known in the industry, some of which are current source drivers. Each of the LED drivers 140/142/144/146 has as an input an analog LED drive signal from the controller DAC 121 (FIG. 1). Each LED driver 140/142/144/146 provides a current (voltage) proportional to the analog LED drive signal that is connected to its corresponding LED 141/143/145/147. The LED 141/143/145/147 will output light at an intensity proportional to this current (voltage). In this embodiment, the light output of each LED is directed toward a filter 150/152/154/156. The LEDs are arranged in order of light output wavelength and, in this example, the filters 150/152/154/156 allow the light from the previous LED to pass through while reflecting light at the wavelength of the filter's 150/152/154/156 corresponding LED. For example, LED1 141 is the highest wavelength and LED4 147 is the lowest wavelength. In other embodiments, LED1 141 is the lowest wavelength and LED4 147 is the highest wavelength. The first filter 150 reflects the light output of LED1 141. The second filter 152 allows light of higher wavelengths than LED 2 143 to pass through it while reflecting wavelength less than or equal to LED 2 143. Therefore, the light from LED1 141, reflected off the first filter 150 passes through the second filter 152 while the light from LED2 143 reflects off of the second filter 152. Each subsequent stage functions similarly. Each filter is angled at approximately 45 degrees from the path of light from the LEDs 141/143/145/147 and aligned to direct the light output from all LEDs into the fiber optic lens 162 and subsequently through the fiber optic cable 164 to the tip of the needle in the patient's venous system (not shown). Before the light output reaches the fiber optic lens 162, a substantially transparent filter 158 directs a very small percentage of the light to the detector 160. The detector 160 is any photo detector capable of measuring light intensity at the wavelengths used the system and outputting an analog signal (voltage, current or impedance) representative of the light power output. The output light power level signal is connected to the input of the ADC 123 of the controller 100. The firmware of the present system periodically samples the output power level from the ADC 123 and adjusts the output levels of the DACs 121 to compensate for any over or under power levels with respect to the user's settings.

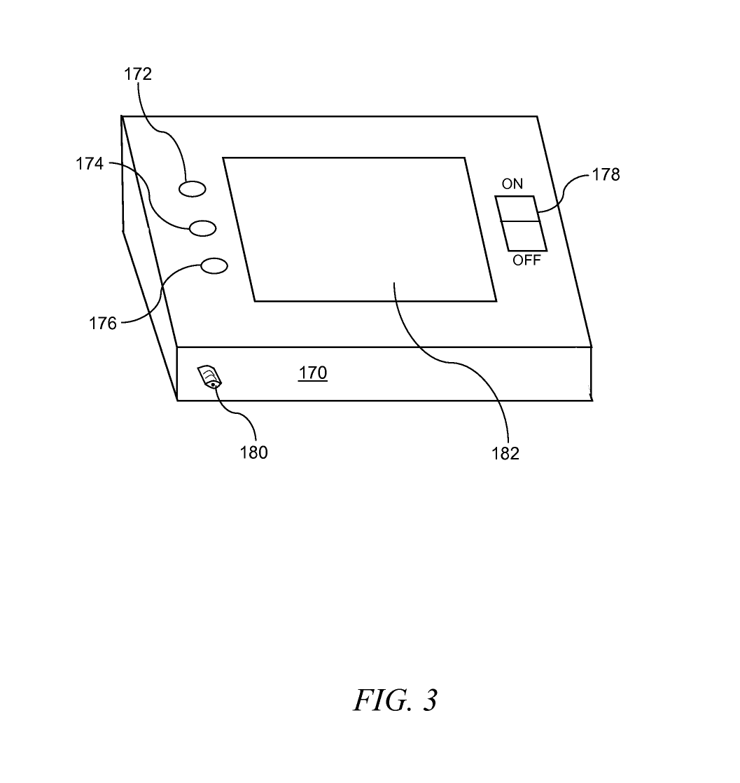

Referring now to FIG. 3, an isometric view of a typical enclosure for the present invention will be described. In this embodiment, an enclosure 170 contains the internal circuitry of the light source of the present invention including the controller 100 and associated input/output subsystems, the LEDs and drivers 141/143/145/147, optics 150/152/154/156/158/162, and detector 160 (all not visible in FIG. 3). Additionally, indicator lamps indicate power on 172 (e.g., green), ultraviolet treatment active 174 (e.g., Blue) and visible light treatment active 176 (e.g., white led). The LCD display and touch screen 182 is preferably located on an upper surface of the enclosure 170. A power switch 178 is provided to turn the system on and off. A fiber optic connector 180 is provided to connect to the fiber optic cable (not shown) that transmits light from the light source of the present invention to the tip of a needle (not shown) that is inserted into the patient's venous system.

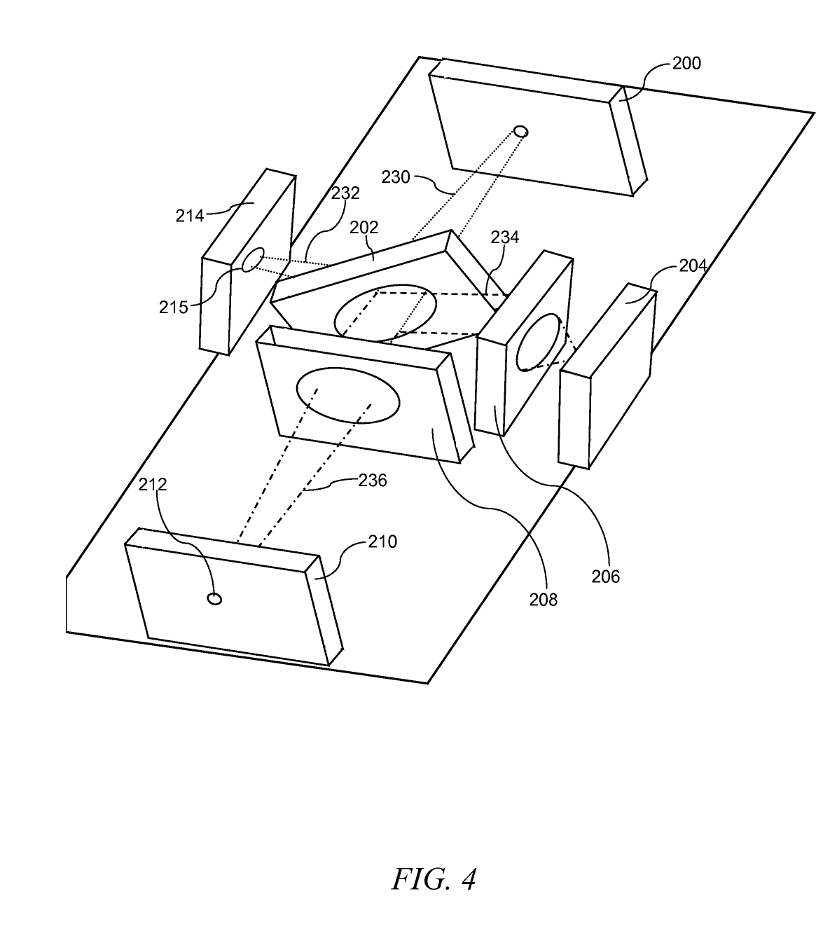

Referring now to FIG. 4, an isometric view of the interrelationship between the light sources, photo detector and fiber optics of the present invention will be described. In this embodiment, multiple ultra violet LEDs are encapsulated into a single package 200 and the ultraviolet light 230 is aimed at a filter 202. The filter 202 passes most of (a majority) the ultraviolet light 230 while reflecting a minimal amount or minority of light 232. The minority of ultraviolet light 230 that does not pass through the filter 202 is reflected 232 onto a photo detector's 214 lens 215. In this way, the photo detector 214 monitors the power output of the ultraviolet light source 200. The majority of the ultraviolet light 230 from the ultraviolet light source 200 mixes with visible light 234 that is emitted from, for example, a white LED 204, focused with a lens 206. The combined ultraviolet and visible light 236 is focused by a lens 208 onto the optics 212 of a fiber optic lens 210 and passed out of the system on a fiber optic cable (not shown). The system of FIG. 4 is one example of how the ultraviolet light and visible light are combined and delivered to the fiber optic. There are many ways known to mix light from different sources and focus the light including lenses, mirrors, filters, prisms and the like and the present invention is not limited to the exemplary embodiment. Furthermore, the system of the present invention is intended to emit any single or combined wavelength of light from one or several of the ultraviolet and visible LEDs.

Equivalent elements can be substituted for the ones set forth above such that they perform in substantially the same manner in substantially the same way for achieving substantially the same result.

It is believed that the system and method of the present invention and many of its attendant advantages will be understood by the foregoing description. It is also believed that it will be apparent that various changes may be made in the form, construction and arrangement of the components thereof without departing from the scope and spirit of the invention or without sacrificing all of its material advantages. The form herein before described being merely exemplary and explanatory embodiment thereof. It is the intention of the following claims to encompass and include such changes.

I claim:

1. A light source, comprising:
one or more light emitting diodes;
one or more filters in radiant communication with the one or more light emitting diodes; and
a fiber optic lens in radiant communication with the one or more filters.

2. The light source of claim 1 wherein the one or more light emitting diodes are arranged in order of light output wavelength relative to the fiber optic lens, the one or more light emitting diodes including a most distal light emitting diode and a most proximal light emitting diode relative to the fiber optic lens.

3. The light source of claim 1 wherein each light emitting diode has a corresponding filter in radiant communication there with.

4. The light source of claim 3 wherein each filter is operationally configured to filter different wavelengths of light.

5. The light source of claim 2 wherein each light emitting diode has a corresponding filter in radiant communication there with, the filters being arranged in like manner as the light emitting diodes including a most distal filter and a most proximal filter relative to the fiber optic lens.

6. The light source of claim 5 wherein each of the non most distal filters are operationally configured to receive light from non-corresponding light emitting diodes and allow the light to pass there through and reflect light received from the non-corresponding light emitting diodes that is of the wavelength for the light emitting diode corresponding to that particular filter.

7. A system for transmitting light to an intravenous needle, comprising:
a light source; and
a fiber optic cable;
wherein the light source includes (1) one or more light emitting diodes, (2) one or more filters in radiant communication with the one or more light emitting diodes, (3) a fiber optic lens in radiant communication with the one or more filters and the fiber optic cable; and
wherein the fiber optic cable is operationally configured to transmit light from the light source to an intravenous needle.
